(19) United States
(12) Patent Application Publication
(10) Pub. No.: US 2020/0197241 A1
(43) Pub. Date: Jun. 25, 2020

(54) UMBILICAL CORD COVER

(71) Applicant: Linda Sinishtaj, Washington, MI (US)

(72) Inventor: Linda Sinishtaj, Washington, MI (US)

(21) Appl. No.: 16/230,301

(22) Filed: Dec. 21, 2018

(51) Int. Cl. A61F 15/00

(52) U.S. Cl. CPC A61F 15/008; A61F 2013/1504

(57) ABSTRACT

An umbilical cord cover includes a fluid-tight covering portion defining a cavity, a contact portion around the covering portion, and a releasable adhesive on the contact portion. The releasable adhesive and the contact portion form a fluid-tight seal around the cavity when the contact portion engages a surface.

### BACKGROUND

Upon severing an umbilical cord, an infant retains a portion of the umbilical cord. This portion is an “umbilical stump.” The umbilical stump is typically 2-3 centimeters (cm) in length and extends from the stomach of the infant. No longer requiring the umbilical cord for nutrients, the umbilical stump dries and falls from the infant within a few weeks.

While the umbilical stump is attached to the infant, the umbilical stump may be a prime site for bacterial colonization and may be subject to infection, e.g., omphalitis. Symptoms of omphalitis include, e.g., redness around the umbilical stump, pus discharged from the umbilical stump, fever, etc. The infant's immune system may be susceptible to the bacterial infection, so reducing the risk for infection is important. In particular, if the umbilical stump gets wet, the umbilical stump may allow for more bacterial growth and the risk for infection increases. This increased risk of infection may increase stress loads for parents of infants.

Parents can take several measures to prevent infection of the umbilical stump. In general, the umbilical stump should remain away from fluids, such as water, that may wet the umbilical stump and delay drying of the umbilical stump, increasing the risk of an infection. Common techniques to dry the umbilical stump include exposing the umbilical stump to air and using clothing with notches that allow air to pass over the umbilical stump.

One method to address umbilical stump infection is to regularly apply an antiseptic to the umbilical stump until the umbilical stump falls off. However, use of antiseptics to avoid infection may delay drying of the umbilical stump. The antiseptic must also be applied several times over the weeks until the umbilical stump falls off, requiring additional steps that may further increase stress load on parents of infants. Furthermore, typical antiseptics, such as alcohol, iodine, and chlorhexidine, may irritate skin around the umbilical stump, causing discomfort for the infant. As a result, using antiseptics alone may not be ideal to reduce the risk of infection.

In general, infants are exposed to dirt and bacteria. Bathing infants removes dirt and bacteria, reducing the risk of infection. However, bathing infants may be challenging while the umbilical stump remains. During a bath, it may be difficult to maintain dryness of the umbilical stump from the water and cleaning agents used during the bath. As immersion would cause the umbilical stump to become wet, sponge baths are typically recommended to avoid fluid contact with the umbilical stump. However, even during sponge baths, incidental fluid contact from bathwater and/or cleaning agents may delay drying of the umbilical stump. For example, movement of the infant during the bath may result in fluid contact with the umbilical stump, e.g., from splashing. The difficulty in maintaining dryness of the umbilical stump may increase stress load on parents of infants. Reducing the opportunity for fluid contact with the umbilical stump while bathing remains a challenge.

While the umbilical stump should remain dry, the infant still requires bathing. This presents a challenge for parents to bathe the infant, which requires contact with water, while keeping the umbilical stump dry. This may increase stress load on parents of infants trying to reduce the risk of bacterial infection. There remains an opportunity to design an apparatus to maintain dryness of the umbilical stump while bathing.

### BRIEF DESCRIPTION OF THE DRAWINGS

FIG. 1 is a perspective view of an umbilical cord cover.

FIG. 2 is a plan view of the umbilical cord cover.

FIG. 3 is a bottom perspective view of the umbilical cord cover.

FIG. 4 is a side cross-sectional view of the umbilical cord cover engaging a surface and covering an umbilical stump.

FIG. 5 is a bottom perspective view of the umbilical cord cover with a film on an adhesive layer.

FIG. 6 is a view of the umbilical cord cover in a tray.

### SUMMARY OF THE INVENTION

An umbilical cord cover includes a fluid-tight covering portion defining a cavity a contact portion around the covering portion, and a releasable adhesive on the contact portion, wherein the releasable adhesive and the contact portion form a fluid-tight seal around the cavity with the contact portion engages a surface.

The contact portion may be annular.

The releasable adhesive may be designed to adhere to skin.

The contact portion may extend out from the covering portion.

The releasable adhesive and the contact portion may form a fluid-tight seal when the contact portion engages a surface.

The covering portion may be silicone.

The releasable adhesive may be designed to maintain an adhesive property upon disengaging from a surface.

The covering portion may extend above the contact portion.

The contact portion may have a lower surface and the releasable adhesive may be disposed on the lower surface. The lower surface of the contact portion may be designed to engage a surface.

The covering portion may be substantially hemispherical

When the contact portion engages a surface, the surface and the covering portion may define a fluid-tight chamber.

The covering portion may have an inner layer and an outer layer. The outer layer may be fluid-resistant.

The cover may further include a coating disposed over an entire surface of the covering portion and the contact portion.

The cover may further include a film attached to the releasable adhesive. The releasable adhesive may be designed to maintain an adhesive property upon removal of the film.

### DETAILED DESCRIPTION

With reference to the Figures, wherein like numerals indicate like parts throughout the several views, an umbilical cord cover 10 includes a fluid-tight covering portion 12, a contact portion 14 around the covering portion 12, and a releasable adhesive 18 on the contact portion 14. The adhesive 18 may be disposed on a lower surface 16 of the contact portion 14.

Upon severing the umbilical cord, the remaining portion of the umbilical cord attached to the infant is an “umbilical stump” 20. The umbilical stump 20 dries and falls off over time. If the umbilical stump 20 becomes wet, e.g., from contact with bathwater, soap, etc., the umbilical stump 20 may provide a site for bacterial colonization, increasing a risk of infection such as omphalitis. That is, the wet umbilical stump 20 may encourage bacterial growth and possible infection. In general, exposing the umbilical stump 20 to air dries the umbilical stump 20 more quickly, and reducing fluid contact from fluids such as bathwater hastens drying of the umbilical stump, reducing the risk of infection.

The umbilical cord cover 10 advantageously protects the umbilical stump 20 from contact with fluids, allowing the umbilical stump 20 to dry and fall off more quickly and reducing risk of infection. For example, while bathing, the umbilical cord cover 10 can protect the umbilical stump 20 from water and cleaning agents, allowing the umbilical stump 20 to remain dry. The umbilical cord cover 10 is releasable from the skin surrounding the umbilical stump while maintaining adhesive properties and may be used multiple times for successive bathing sessions until the umbilical stump 20 falls off. Thus, parents can protect the umbilical stump 20 from fluids while bathing, reducing the risk of infection. Thus, the umbilical cord cover 10 eases the minds of parents and allows them to focus on other aspects of caring for the infants.

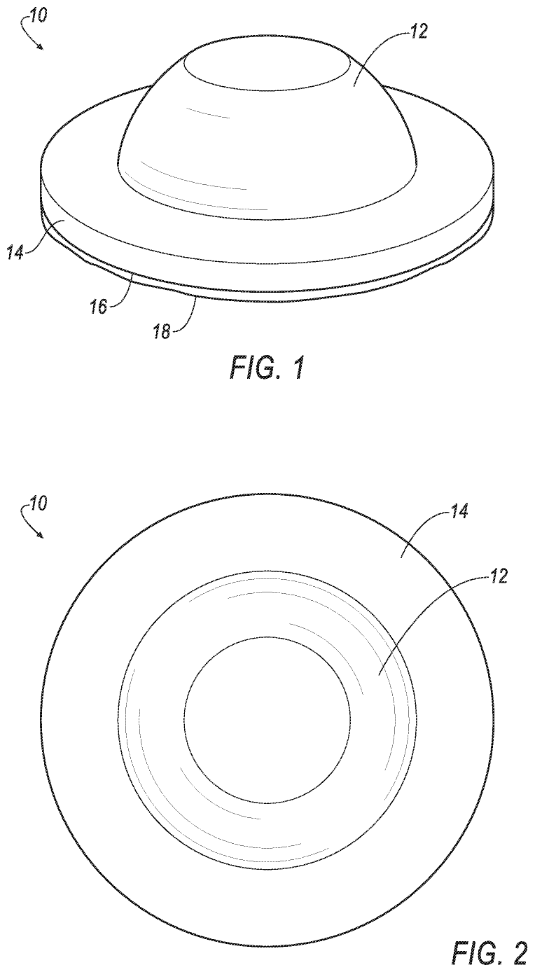

As shown in FIGS. 1-6, the umbilical cord cover 10 includes the covering portion 12. The covering portion 12 covers an umbilical stump 20. The covering portion 12 protects the umbilical stump 20 from contact with fluids. The covering portion 12 extends away from the umbilical stump 20, blocking fluids from reaching the umbilical stump 20, e.g. during bathing. The covering portion 12 may be substantially hemispherical. Alternatively, the covering portion 12 may be any suitable shape to cover the umbilical stump, e.g., conical, cylindrical, parabolic, etc.

The covering portion 12 is fluid-tight. That is, the covering portion 12 resists intrusion from fluids, e.g., water, oil, etc. The covering portion 12 may be a unitary construction having no holes, gaps, slits, etc., that may allow fluid to pass through the covering portion 12. The covering portion 12 may be a fluid-resistant material, e.g., silicone, Teflon®, or any other suitable material. For example, the covering portion 12 may be food-grade silicone. The food-grade silicone is nonpermeable, i.e., resists entry of fluids such as water, protecting the umbilical stump from fluids. Furthermore, food-grade silicone has no known allergic reaction. The covering portion 12 may be substantially rigid. Alternatively, the covering portion 12 may be resilient or flexible. The covering portion 12 prevents water from contacting the umbilical stump 20 while bathing, allowing the umbilical stump 20 to dry more quickly and reducing the risk of infection.

The covering portion 12 may include a fluid-resistant coating. For example, the covering portion 12 may include a hydrophobic and/or oleophobic coating that reduces or prevents fluids from adhering and/or wetting to the covering portion 12. The fluid-resistant coating may be, e.g., Teflon®, polysiloxane, polystyrene, wax, polyester, silane, or any other suitable material. The fluid-resistant coating may cause fluids to reduce surface tension with the covering portion 12, increasing a contact angle with the covering portion 12 and causing fluids to roll off the covering portion 12 more readily. The coating may be disposed over an entire surface of the covering portion 12 and the contact portion 14. The coating may be a liquid that is applied to the covering portion 12 and/or the contact portion 14, when then dries to form the fluid-resistant coating. Alternatively, the coating may be a solid, e.g., a shrink-wrapped polymer, that is placed on the covering portion 12 and/or the contact portion 14. For example, the coating may be a shrink-wrapped plastic film that is wrapped onto the covering portion 12 and then conformed onto the covering portion 12 by applying heat.

The covering portion 12 may be a multi-layer construction. The covering portion 12 may have an inner layer and an outer layer. For example, the inner layer may be a solid polymer, e.g., food-grade silicone, and the outer layer may be a fluid-resistant coating, e.g., a hydrophobic polysiloxane. The solid polymer provides strength to the umbilical cord cover 10, and the fluid-resistant coating repels fluid. In another example, the outer layer may be food-grade silicone, and the inner layer may be a fluid-absorbent polymer, e.g., a hydrogel. The silicone repels fluid outside of the umbilical cord cover 10, and the fluid-absorbent polymer absorbs fluid that may otherwise reach the umbilical stump 20. The covering portion 12 may have a plurality of fluid-resistant and fluid-absorbent layers depending on the desired repellant and absorbent characteristics to protect the umbilical stump 20 from fluid contact.

The covering portion 12 resists cleaning agents, e.g., soaps, detergents, etc. During bathing, the umbilical cord cover 10 may contact cleaning agents dispersed in the water. The covering portion 12 may be constructed of a material that prevents entry of these cleaning agents, e.g., silicone, Teflon®, etc. By resisting the cleaning agents, the covering portion 12 prevents the cleaning agents from contacting the umbilical stump 20, allowing the umbilical stump 20 to stay dry. Thus, the covering portion 12 prevents water and other fluids from contacting the umbilical stump 20.

The covering portion 12 defines a cavity 22. The cavity 22 receives the umbilical stump 20. The cavity 22 may be any suitable shape. For example, the covering portion 12 may be substantially hemispherical and may define a hemispherical cavity 22. Alternatively, the covering portion 12 may be substantially cylindrical and may define a cylindrical cavity 22. The cavity 22 is designed to extend above the umbilical stump 20 when the umbilical cord cover engages a surface 24, e.g., skin around the umbilical stump 20, as shown in FIG. 4. The cavity 22 may be sized to accommodate umbilical stumps 20 of different sizes such that a single design of the umbilical cord cover 10 may be used. For example, the cavity 22 may be 5 cubic centimeters.

As shown in FIGS. 1-6, the umbilical cord cover 10 includes the contact portion 14. The contact portion 14 contacts the surface 24, e.g., skin around the umbilical stump 20. The contact portion 14 secures the covering portion 12 to the surface 24. The contact portion 14 may be a fluid-resistant material, e.g., silicone, Teflon®, or any other suitable material. For example, the contact portion 14 may be food-grade silicone that resists entry of fluids, as described above. Because the contact portion 14 contacts the surface 24, the contact portion 14 may be a non-irritating material such as food-grade silicone. The contact portion 14 and the covering portion 12 may be the same material, e.g., food-grade silicone. Alternatively, the contact portion and the covering portion 12 may be different materials. The contact portion 14 may be substantially rigid. Alternatively, the contact portion 14 may be resilient or flexible.

The contact portion 14 resists cleaning agents, e.g., soaps, detergents, etc. The contact portion 14 may be constructed of a material that prevents entry of these cleaning agents, e.g., silicone, Teflon®, etc. By resisting the cleaning agents, the contact portion 14 prevents the cleaning agents from contacting the umbilical stump 20, allowing the umbilical stump 20 to stay dry. Thus, the contact portion 14 and the covering portion 12 prevent water and other fluids from contacting the umbilical stump 20.

The contact portion 14 may be a unitary construction with the covering portion 12 of a single material, e.g., food-grade silicone. As a unitary construction, the contact portion 14 and the covering portion 12 may advantageously resist entry from fluids because the connection between the contact portion 14 and the covering portion 12 lacks any gaps, holes, seams, etc., through which fluid may pass. That is, the molecular bonds in the unitary construction may be stronger than an equivalent assembled construction with an intermediate fastener, providing additional protection for the umbilical stump 20 from fluids. The contact portion 14 and the covering portion 12 may be, e.g., injection-molded, extruded, additively printed, etc.

Alternatively, the contact portion 14 and the covering portion 12 may be separately formed pieces connected by any suitable fastener, e.g., an adhesive, a weld, a shrink-wrapped cover, etc. The fastener may be fluid-tight to prevent the umbilical stump 20 from contacting fluid. Constructing the contact portion 14 and the covering portion 12 separately and later joining the contact portion 14 to the covering portion 12 may ease manufacturing. When the contact portion 14 and the covering portion 12 are separately formed pieces, the contact portion 14 and the covering portion 12 may be different materials, e.g., different polymers.

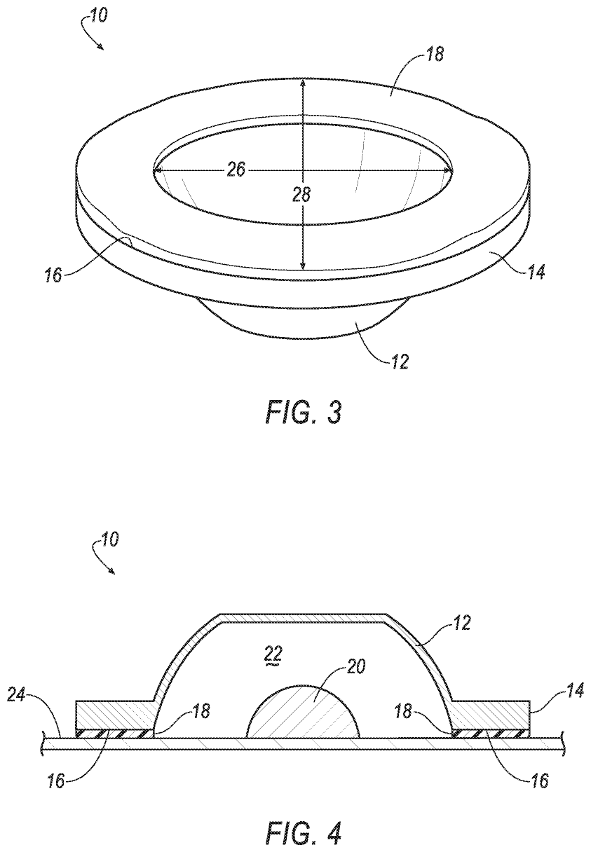

As shown in FIGS. 1-6, the contact portion 14 may be annular. That is, the contact portion 14 may be ring-shaped, defining an inner diameter 26 and an outer diameter 28, as shown in FIG. 3. Alternatively, the contact portion 14 may have a different shape, e.g., concentric ellipses, differing inner and outer shapes, polygons, etc. The annular shape of the contact portion 14 allows the umbilical cord cover 10 to define the cavity 22 while maintaining contact with the surface. The inner diameter 26 and the outer diameter 28 may be chosen to specify the size of the contact portion 14. For example, the inner diameter 26 may be 4 centimeters and the outer diameter 28 may be 5 centimeters. The shape and size of the contact portion 14 and the cover portion 12 may be determined to ensure space in the cavity 22 around the umbilical stump 20, i.e., such that no part of the umbilical cord cover 10 contacts the umbilical stump 20. That is, contact with the umbilical stump 20 may delay drying of the umbilical stump 20. Furthermore, contact with the umbilical stump 20 may cause discomfort for the infant. Thus, the cavity 22 may have enough space to remain spaced from the umbilical stump 20 even with incidental contact with the umbilical cord cover 10.

The contact portion 14 has a lower surface 16 that engages the surface 24, e.g., the skin. The lower surface 16 maintains contact with the surface 24, preventing fluid from entering the cavity 22. When the contact portion 14 is annular, the lower surface 16 is annular. The lower surface 16 may be designed to maintain contact with the surface 24 when the surface 24 engages fluids, e.g., during bathing. As shown in FIG. 4, the lower surface 16 is spaced from the umbilical stump 20, i.e., the lower surface 16 is separated from the umbilical stump 20 such that the lower surface 16 does not contact the umbilical stump 20. The lower surface 16 may be substantially flat to maximize contact with the surface 24.

The covering portion 12 extends above the contact portion 14. That is, at least part of the covering portion 12 extends above the contact portion 14 when the contact portion 14 engages the surface 24, as shown in FIG. 4. The covering portion 12 above the contact portion 14 allows the cavity 22 to receive the umbilical stump 20. The covering portion 12 may extend above the contact portion 12 by a specified height. The height may be determined based on, e.g., umbilical stump 20 sizes. The height may be, e.g., 5 centimeters.

When the contact portion 14 engages the surface 24, the cavity 22 is fluid-tight. That is, the contact portion 14 and the surface 24 prevent fluid from entering the cavity 22 and contacting the umbilical stump 20. Thus, the umbilical stump 20 remains dry when within the cavity 22.

The umbilical cord cover 10 includes the adhesive 18. The adhesive 18 may be disposed on the contact portion 14. The adhesive 18 secures the contact portion 14 to the surface 24, holding the covering portion 12 over the umbilical stump 20 as shown in FIG. 4. Thus, even when the surface 24 moves away from a flat position, the umbilical cord cover 10 remains fixed to the surface 24. The adhesive 18 is designed to adhere to the surface 24, i.e., to skin. The adhesive 18 may be, e.g., latex, glue, cement, rubber, silicone, polymer, polyurethane, hydrocolloid, or any other suitable chemical with suitable adhesive properties. Such adhesives may be skin-safe, i.e., having little to no adverse reactions to skin.

For example, the adhesive 18 may be a hydrocolloid. The hydrocolloid is a nontoxic chemical that resists fluids such as water, cleaning agents, and bodily fluids. The hydrocolloid may have pressure-sensitive adherence, i.e., applying additional pressure to the hydrocolloid adhesive 18 increases the adhesion strength of the hydrocolloid adhesive 18. The hydrocolloid may adhere quickly, forming a fluid-tight seal between the contact portion 14 and the surface 24.

The hydrocolloid may be fluid-absorbent, absorbing fluids before reaching the umbilical stump 20. That is, the hydrocolloid may have absorbent particulates dispersed throughout to absorb the fluids. The ratio of hydrocolloid to absorbent particulates can be determine to reach a specified adhesion strength and fluid absorption. The hydrocolloid may include a tackifier to increase cohesion strength, increasing the strength of the adhesive 18.

Another example adhesive 18 is a cold seal adhesive. Cold seals are cohesive seals, i.e., they stick to themselves, the contact portion 14, and the surface 24, but not to other adhesives 18 or polymer substrates. Cold seals may be useful to simplify adhesive choice and packaging.

The adhesive 18 may be applied as a liquid directly to the contact portion 14, which then dries to form an adherent layer. Alternatively, the adhesive 18 may be affixed to an intermediate layer, e.g., double-sided tape, prior to application to the contact portion 14.

The adhesive 18 may be releasable, i.e., upon adhering the contact portion 14 to the surface 24, the adhesive 18 may release from the surface 24 when force is applied. For example, when the umbilical cord cover 10 is pulled, the adhesive 18 may release from the surface 24, allowing the umbilical cord cover 10 to be removed from the surface 24. The adhesive 18 may have an adhesion strength designed to secure the contact portion 14 to the surface 24 upon receiving smaller forces, e.g., from fluids, incidental contact with a sponge or towel, etc., and to release the contact portion 14 from the surface 24 upon receiving larger forces, e.g., from manual pulling. Thus, the umbilical cord cover 10 may be secured during bathing and manually removable after bathing. As used herein, an “adhesion strength” is a force required to release the adhesive 18 from the surface 24.

Upon release from the surface 24, the adhesive 18 may retain adhesion, i.e., the adhesive 18 may adhere to another surface 24 after release from the surface 24. Thus, the umbilical cord cover 10 may be used multiple times while maintaining adhesion to the surface 24 each time, e.g., for each bath time, to protect the umbilical stump 20 from fluid. That is, the releasable adhesive 18 may be designed to maintain an adhesive property upon disengaging from the surface 24. The “adhesive property” may be, e.g., a stickiness, a viscosity, an adhesion strength, a cohesion strength, etc.

The adhesive 18 may be fluid-resistant. That is, the adhesive 18 may maintain adhesion to the surface 24 when contacting a fluid, e.g., water, cleaning agents, etc. The adhesive 18 may form a barrier between the surface 24 and the contact portion 14, preventing fluid from entering the cavity 22. The adhesive 18 maintains adhesion to the surface 24 even when contacting fluid. That is, even when the umbilical cord cover 10 contacts fluids, the adhesive 18 maintains adhesion between the contact portion 14 and the surface 24, preventing fluid from entering the cavity 22 and contacting the umbilical stump 20.

The adhesive 18 may resist cleaning agents. That is, the adhesive 18 may be designed to resist common cleaning agents, e.g., soaps, detergents, shampoos, etc. The adhesive 18 may be unreactive with the cleaning agents, retaining adhesive properties even with incidental contact with the cleaning agents. Thus, the adhesive 18 may be used a plurality of times while retaining its adhesive properties.

The adhesive 18 is disposed on the lower surface 16 of the contact portion 14. The lower surface 16 is designed to engage the surface 24. The lower surface 16 and the adhesive 18 contact the surface 24, forming a fluid-tight seal preventing fluid from entering the cavity 22 and preventing the umbilical stump 20 from fluid.

Upon pressing the umbilical cord cover 10 onto the surface 24, the adhesive 18 adheres to the surface 24 to form a fluid-tight seal around the cavity 22. That is, the cavity 22, the adhesive 18, the contact portion 14, the covering portion 12, and the surface 24 define a fluid-tight chamber to cover the umbilical stump 20. Thus, the umbilical cord cover 10 protects the umbilical stump 20 from contact with fluids during bathing. When the bathing session is complete, the umbilical cord cover 10 may be removed until the next bathing session, at which point the umbilical cord cover 10 may be adhered again to the surface 24 to protect the umbilical stump 20.

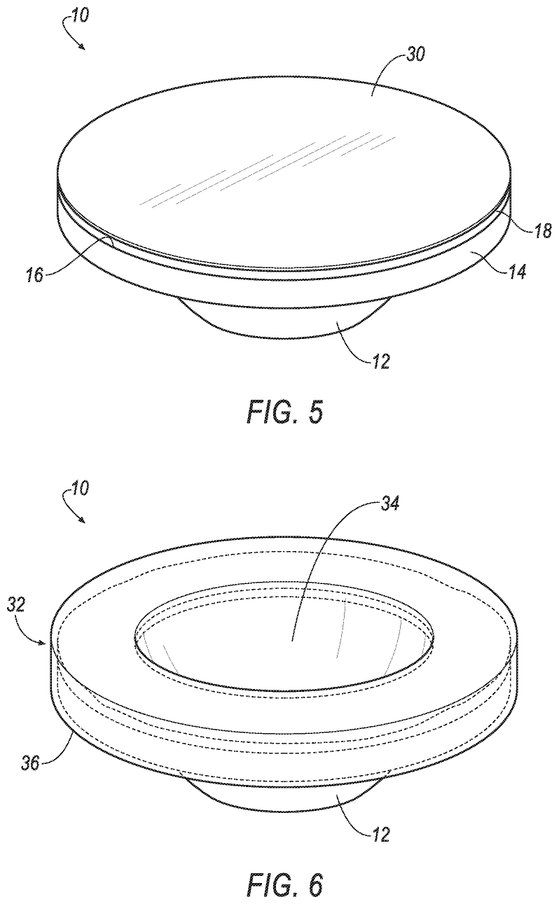

The umbilical cord cover 10 may include a film 30, as shown in FIG. 5. The film 30 may be attached to the adhesive 18. The film 30 may protect the adhesive 18 prior to use of the umbilical cord cover 10. That is, exposure to air may reduce the adhesive properties of the adhesive 18, e.g., the adhesion strength. Reducing the adhesive properties would cause the adhesive 18 to adhere more weakly to the surface 24, possibly allowing fluid to contact the umbilical stump 20. The film 30 protects the adhesive 18 from contact with the air, preserving the adhesive properties of the adhesive 18. The film 30 protects the adhesive 18 prior to use. When the umbilical cord cover is ready for use, the parent removes the film 30. Upon removal of the film 30, the adhesive 18 may then be pressed to the surface 24, securing the contact portion 14 to the surface 24.

The umbilical cord cover 10 may mate with a tray 32, as shown in FIG. 6. The tray 32 may be shaped to conform to the shape of the umbilical cord cover 10. That is, the tray 32 may include a protrusion 34 that enters the cavity 22 and a lip 36 that surrounds the contact portion 14. Thus, the tray 32 may store the umbilical cord cover 10 when not in use. The protrusion 34 may extend into the cavity 22 to prevent the covering portion 12 from collapsing into the cavity 22. The lip 36 may be spaced from the lower surface 16 of the contact portion 14 such that the contact portion 14 does not adhere to the lip 36, preserving the adhesive properties of the adhesive 18. The tray 32 may be used in packaging the umbilical cord cover 10, e.g., to reduce overall packaging material, to protect the adhesive 18, etc.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. An umbilical cord cover, comprising:
a fluid-tight covering portion defining a cavity;
a contact portion around the covering portion; and
a releasable adhesive on the contact portion;
wherein the releasable adhesive and the contact portion form a fluid-tight seal around the cavity when the contact portion engages a surface.

2. The cover of claim 1, wherein the contact portion is annular.

3. The cover of claim 1, wherein the releasable adhesive is designed to adhere to skin.

4. The cover of claim 1, wherein the contact portion extends out from the covering portion.

5. The cover of claim 1, wherein the releasable adhesive and the contact portion form a fluid-tight seal when the contact portion engages a surface.

6. The cover of claim 1, wherein the covering portion is silicone.

7. The cover of claim 1, wherein the releasable adhesive is designed to maintain an adhesive property upon disengaging from a surface.

8. The cover of claim 1, wherein the covering portion extends above the contact portion.

9. The cover of claim 1, wherein the contact portion has a lower surface and the releasable adhesive is disposed on the lower surface.

10. The cover of claim 9, wherein the lower surface of the contact portion is designed to engage a surface.

11. The cover of claim 1, wherein the covering portion is substantially hemispherical.

12. The cover of claim 1, wherein, when the contact portion engages a surface, the surface and the covering portion define a fluid-tight chamber.

13. The cover of claim 1, wherein the covering portion has an inner layer and an outer layer.

14. The cover of claim 13, wherein the outer layer is fluid-resistant.

15. The cover of claim 1, further comprising a coating disposed over an entire surface of the covering portion and the contact portion.

16. The cover of claim 1, further comprising a film attached to the releasable adhesive.

17. The cover of claim 16, wherein the releasable adhesive is designed to maintain and adhesive property upon removal of the film.
